(19) United States
(12) Patent Application Publication
(10) Pub. No.: US 2025/0288225 A1
(43) Pub. Date: Sep. 18, 2025

(54) NON-INVASIVE CONTINUOUS BLOOD GLUCOSE CONCENTRATION (BGC) MEASUREMENT METHOD AND APPARATUS BASED ON OPTICAL COHERENCE TOMOGRAPHY ANGIOGRAPHY (OCTA)

(71) Applicant: ZHEJIANG UNIVERSITY, Hangzhou (CN)

(72) Inventors: Peng Li, Hangzhou (CN); Mengqin Gao, Hangzhou (CN); Jiahao Wang, Hangzhou (CN)

(73) Assignee: ZHEJIANG UNIVERSITY, Hangzhou (CN)

(21) Appl. No.: 19/221,606

(22) Filed: May 29, 2025

Related U.S. Application Data
(63) Continuation-in-part of application No. PCT/CN2024/116734, filed on Sep. 4, 2024.

(30) Foreign Application Priority Data
Sep. 12, 2023 (CN) 202311170515.1

(51) Int. Cl. A61B 5/145; A61B 5/00; A61B 5/026; A61B 5/1455; A61B 5/1495; A61B 90/00

(52) U.S. Cl. CPC A61B 5/14532; A61B 5/0261; A61B 5/1455; A61B 5/1495; A61B 5/7203; A61B 5/7278; A61B 5/1451; A61B 2090/3735; A61B 2562/0242

(57) ABSTRACT

A non-invasive continuous blood glucose concentration (BGC) measurement method and apparatus based on optical coherence tomography angiography (OCTA) are provided. The method includes: performing continuous OCT scanning and imaging on a target tissue region; extracting, based on a depth attenuation characteristic of an OCT signal, a three-dimensional optical scattering coefficient of the target tissue region; generating a three-dimensional microvascular distribution of the target tissue region; accurately distinguishing a blood optical scattering coefficient (BOC) from a tissue optical scattering coefficient (TOC) based on the three-dimensional scattering coefficient and the three-dimensional microvascular distribution; and measuring reference BGCs at two time points through an electrochemical method; and combining the reference BGCs with the BOC and the TOC respectively, thereby achieving continuous measurement of a BGC and an interstitial fluid glucose concentration (IGC). The method and apparatus are based on OCTA to achieve accurate BGC measurement while simultaneously enabling IGC measurement.

### CROSS REFERENCE TO THE RELATED APPLICATIONS

This application is a continuation-in-part application of International Application No. PCT/CN2024/116734, filed on Sep. 4, 2024, which is based upon and claims priority to Chinese Patent Application No. 202311170515.1, filed on Sep. 12, 2023, the entire contents of which are incorporated herein by reference.

### TECHNICAL FIELD

The present disclosure belongs to the field of biomedical detection, and relates to a blood glucose concentration (BGC) measurement method and apparatus based on optical coherence tomography angiography (OCTA), in particular to a non-invasive continuous BGC measurement method and apparatus based on OCTA.

### BACKGROUND

Diabetes is a chronic metabolic disorder that can lead to serious complications such as heart disease, kidney problems, and stroke. Monitoring blood glucose levels is crucial for patients' overall health. Glucose levels are typically assessed in two biological fluids: blood and interstitial fluid. Blood glucose concentrations (BGCs) are usually measured using enzyme-based electrochemical apparatuses that require blood samples collected through finger or forearm pricks, which may cause discomfort, infection risks, and poor compliance. An approach for continuously monitoring interstitial fluid glucose concentrations (IGCs) involves subcutaneous needle-type sensors. However, this approach demonstrates lower accuracy compared to blood glucose monitoring due to the delayed response of interstitial fluid glucose levels and potential skin irritation.

Optical techniques utilizing near-infrared light scattering hold significant potential for non-invasive glucose monitoring. However, detecting glucose-induced weak scattering variations within a strong scattering background remains a major challenge for blood glucose testing. Although optical coherence tomography (OCT) can identify scattering changes caused by glucose in specific tissue layers, distinguishing between blood and interstitial fluid still poses a substantial obstacle. Blood glucose and interstitial fluid glucose exert opposite effects on the optical scattering properties of surrounding tissues, which may significantly impact the accuracy of BGC measurements.

Optical coherence tomography angiography (OCTA) enables three-dimensional capillary-level vascular mapping of blood perfusion. However, distinguishing between blood and interstitial fluid using OCTA and subsequently achieving BGC measurements remains a challenge for those skilled in the art.

### SUMMARY

In response to the problems in the prior art, an objective of the present disclosure is to propose a non-invasive continuous blood glucose concentration (BGC) measurement method and apparatus based on optical coherence tomography angiography (OCTA). The present disclosure leverages an OCTA vascular mask to distinguish a blood optical scattering coefficient (BOC) from a tissue optical scattering coefficient (TOC), thereby precisely measuring a glucose-induced scattering variation in a blood or interstitial fluid region. Given a linear relationship between the optical scattering coefficient and the glucose concentration, OCTA holds potential to achieve high-precision, non-invasive, and continuous blood glucose measurement.

The present disclosure adopts the following technical solutions.

### I. Non-Invasive Continuous BGC Measurement Method Based on OCTA

A continuous OCTA imaging method includes: performing, by an OCT apparatus, continuous OCT scanning and imaging on a target tissue region; and obtaining, by a processor, an OCT signal.

A calculation method for optical scattering coefficients of blood and a surrounding tissue includes: generating, by the processor, a three-dimensional optical scattering coefficient and a three-dimensional microvascular distribution of the target tissue region based on the OCT signal; and separately calculating a blood optical scattering coefficient (BOC) and a tissue optical scattering coefficient (TOC) based on the three-dimensional optical scattering coefficient and the three-dimensional microvascular distribution.

A BGC calibration and calculation method includes: collecting a blood sample, and measuring reference BGCs at two time points of t1 and t2 through an electrochemical method; and performing, by the processor, data pairing and linear fitting on the reference BGC and a BOC at a same time point, thereby obtaining BGCs at all the time points.

An interstitial fluid glucose concentration (IGC) calibration and calculation method includes: performing, by the processor, data pairing and linear fitting on the reference BGCs at the two time points of t1 and t2 and corresponding TOCs delayed by ΔT, thereby obtaining IGCs at all the time points.

A non-invasive diabetes detection method includes: extracting, by the processor, features of an optical BGC curve and an optical IGC curve over time, respectively, based on the BGCs and the IGCs at all the time points, and detecting diabetes based on the features of the optical BGC curve and the optical IGC curve over time.

The continuous OCTA imaging method is selected from one of the following methods:
a time-domain OCT imaging method that alters an optical path length of a reference arm through scanning;
alternatively, a spectral-domain OCT imaging method that records a spectral interference signal through a spectrometer; and
alternatively, a swept-source OCT imaging method that records a spectral interference signal through a swept-source.

The calculation method for optical scattering coefficients of blood and a surrounding tissue specifically includes:
extracting, based on a depth attenuation characteristic of OCT, a three-dimensional optical scattering coefficient of the target tissue region from the OCT signal;
generating a three-dimensional microvascular distribution of the target tissue region from the OCT signal; and
subjecting the three-dimensional microvascular distribution to image binarization, and obtaining a vascular mask and a tissue mask; and subjecting the three-dimensional optical scattering coefficient to point-wise multiplication with the vascular mask and the tissue mask separately, and obtaining a BOC and a TOC.

The extracting, based on a depth attenuation characteristic of OCT, a three-dimensional optical scattering coefficient of the target tissue region from the OCT signal specifically includes:
first, removing system noise in a depth direction of the OCT signal, and obtaining a denoised OCT signal;
next, compensating a depth-direction attenuation in the denoised OCT signal through a Gaussian fitting model, and obtaining a compensated OCT signal; and
finally, calculating the optical scattering coefficient of the target tissue region according to a depth-direction variation feature of the compensated OCT signal.

The generating a three-dimensional microvascular distribution of the target tissue region from the OCT signal specifically includes: analyzing, by an OCT blood flow signal extraction method, OCT signal amplitude or phase, or both amplitude and phase, and obtaining the three-dimensional microvascular distribution of the target tissue region.

The OCT blood flow signal extraction method includes: difference calculation, speckle variance operation, decorrelation calculation, or eigenvalue decomposition calculation.

The subjecting the three-dimensional microvascular distribution to image binarization, and obtaining a vascular mask and a tissue mask; and subjecting the three-dimensional optical scattering coefficient to point-wise multiplication with the vascular mask and the tissue mask separately, and obtaining a BOC and a TOC specifically includes:
setting pixel values of all blood flow regions in the three-dimensional microvascular distribution to 1 and pixel values of non-blood-flow regions to 0; obtaining the vascular mask; subjecting the vascular mask to point-wise multiplication with the three-dimensional optical scattering coefficient; and extracting the BOC; and
setting pixel values of all blood flow regions in the three-dimensional microvascular distribution to 0 and pixel values of non-blood-flow regions to 1; obtaining the tissue mask; subjecting the tissue mask to point-wise multiplication with the three-dimensional optical scattering coefficient; and extracting the TOC.

### II. A Non-Invasive Continuous BGC Measurement Apparatus Based on OCTA Includes:

the OCT apparatus, configured to perform continuous OCT imaging on the target tissue region; and
one or more processors, configured to perform dynamic real-time data processing.

The OCT apparatus is selected from one of:
an OCT apparatus including: a low-coherence light source, an interferometer, and a detector;
alternatively, an OCT apparatus including: a low-coherence light source, an interferometer, and a spectrometer;
alternatively, an OCT apparatus including: a swept broadband light source, an interferometer, and a detector; and
alternatively, any other apparatus able to perform OCT.

The present disclosure has the following beneficial effects:

The present disclosure is based on OCTA to achieve accurate BGC measurement while simultaneously enabling IGC measurement. Compared to existing continuous non-invasive blood glucose monitoring technologies, the present disclosure exhibits superior measurement accuracy and sensitivity, demonstrating significant implications for advancing optical continuous non-invasive blood glucose monitoring. Within Parke's error grid analysis, all measurement results of the present disclosure are located in clinically acceptable Zones A+B, with 96.69% of values falling within Zone A. Furthermore, 94.21% of the measurement results in the present disclosure comply with the International Organization for Standardization (ISO) accuracy standards for glucose monitoring.

### BRIEF DESCRIPTION OF THE DRAWINGS

FIG. 1 is a schematic diagram of a method according to the present disclosure;

FIG. 2 is a schematic diagram of an apparatus according to the present disclosure;

FIG. 3 is a schematic diagram of an embodiment of the present disclosure;

FIG. 4 is a schematic diagram of a blood glucose test process according to the present disclosure;

FIGS. 5A-5C show schematic diagrams of a three-dimensional OCT structure, a three-dimensional optical scattering coefficient, and three-dimensional OCTA according to an embodiment of the present disclosure;

FIGS. 6A-6E show typical OCTA images according to an embodiment of the present disclosure; and

FIGS. 7A-7D show relationship plots over time between an optical BGC, an optical IGC, and a reference BGC obtained using the method of the present disclosure during an oral glucose tolerance test (OGTT) according to an embodiment of the present disclosure.

FIGS. 8A-8B show accuracy of non-invasive BGC measurement based on OCTA in an embodiment of the present disclosure.

Reference Numerals: 1. continuous OCTA imaging method; 2. calculation method for optical scattering coefficients of blood and surrounding tissue; 3. BGC calibration and calculation method; 4. IGC calibration and calculation method; 11. light source; 12. beam splitter; 13. reference arm collimator; 14. planar high-reflectivity mirror; 15. sample arm collimator; 16. scanning galvanometer; 17. objective lens; 18. test sample; 19. interference signal detection apparatus; 20. signal processor; 21. extract a three-dimensional optical scattering coefficient of a target tissue region from an OCT signal based on a depth attenuation characteristic; 22. generate a three-dimensional microvascular distribution of the target tissue region from the OCT signal; 23. binarize the three-dimensional microvascular distribution to obtain vascular and tissue masks, and subjecting the three-dimensional optical scattering coefficient to point-wise multiplication with the vascular and tissue masks, thereby obtaining a BOC and a TOC; 24. polarization controller; 31. broadband swept-source; 32. 80:20 fiber coupler; 33. 50:50 fiber coupler; 34. first optical circulator; 35. second optical circulator; 36. polarization controller; 40. first collimator; 37. second collimator; 38. focusing lens; 42. scanning lens; 39. planar high-reflectivity mirror; 41. scanning galvanometer; 43. sample; 44. balanced detector; and 45. processor.

### DETAILED DESCRIPTION OF THE EMBODIMENTS

The solutions of the present disclosure will be described in further detail below with reference to the drawings, which constitute a part of the present disclosure. It should be noted that these description and examples are merely intended to specifically explain the present disclosure, rather than the limit the present disclosure. Any modifications and changes made to the present disclosure within the spirit and the protection scope of the claims of the present disclosure should fall within the protection scope of the present disclosure.

Meanwhile, to facilitate understanding of the embodiments of the present disclosure, the operations are described as multiple discrete operations, but the order of description does not represent the order in which the operations are implemented.

In the description in the embodiments of the present disclosure, a sample measurement space is represented by an x-y-z three-dimensional coordinate system based on spatial directions, and variables are denoted by letters such as I, H, and m. This representation is only intended to facilitate discussion and does not limit the application of the present disclosure. I, H, and m may take any numerical values such as 1, 2, or 3.

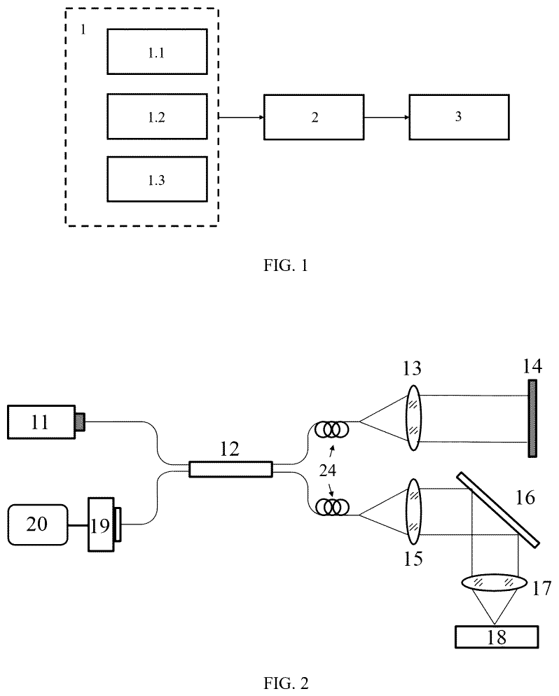

A method of the present disclosure is shown in FIG. 1. Continuous OCTA imaging method 1 is used to perform continuous OCT scanning and imaging on a target tissue region to obtain an OCT signal.

The continuous OCTA imaging method 1 is selected from one of the following methods:
a time-domain OCT imaging method that alters an optical path length of a reference arm through scanning;
alternatively, a spectral-domain OCT imaging method that records a spectral interference signal through a spectrometer; and
alternatively, a swept-source OCT imaging method that records a spectral interference signal through a swept-source.

Calculation method 2 for optical scattering coefficients of blood and a surrounding tissue is used to generate a three-dimensional optical scattering coefficient and a three-dimensional microvascular distribution of a target tissue region from an OCT signal, and to separately calculate blood optical scattering coefficient (BOC) and tissue optical scattering coefficient (TOC) based on the three-dimensional optical scattering coefficient and the three-dimensional microvascular distribution.

The calculation method 2 for optical scattering coefficients of blood and a surrounding tissue specifically includes the following step.

S21, three-dimensional optical scattering coefficient of the target tissue region is extracted from the OCT signal based on a depth attenuation characteristic of OCT.

Specifically, S21, the three-dimensional optical scattering coefficient of the target tissue region is extracted from the OCT signal based on the depth attenuation characteristic of OCT as follows.

First, system noise in a depth direction of the OCT signal is removed to obtain a denoised OCT signal.

Next, a depth-direction attenuation in the denoised OCT signal is compensated through a Gaussian fitting model to obtain a compensated OCT signal.

Finally, the optical scattering coefficient of the target tissue region is calculated according to a depth-direction variation feature of the compensated OCT signal.

### More Specifically:

First, the system noise in the depth direction of the OCT signal is removed to obtain the denoised OCT signal. Subsequently, the depth-direction attenuation in the denoised OCT signal is compensated through the Gaussian fitting model to obtain the compensated OCT signal. Finally, the optical scattering coefficient of the target tissue region is calculated according to the depth-direction variation feature of the compensated OCT signal.

In the above operation, the denoising operation is as follows:

$$I ( z ) = {I}_{inital} ( z ) - N ( z ) ;$$

where, Iᵢₙᵢₜₐₗ denotes an original OCT signal intensity at depth position z; N (z) denotes average system noise at the depth position z; and I(z) denotes a denoised OCT signal intensity at the depth position z. The extraction of the average system noise is as follows. First, the system noise is collected by blocking a sample arm of a blood glucose test apparatus. Then, the system noise at each XY plane is averaged along the depth direction to obtain one-dimensional averaged system noise distributed along the depth direction.

The compensation operation is as follows:

$${I}_{c orrect} = \frac{I ( z )}{S ( z )} ;$$

where, I_{correct} denotes the compensated OCT signal intensity; and S(z) denotes the Gaussian fitting model obtained through Gaussian fitting of the OCT signal intensity attenuation along the depth direction.

$$S ( z ) = exp ( - {z}^{2} / {σ}^{2} ) ;$$

σ denotes an exponential attenuation parameter of the OCT signal intensity in the depth direction, specifically measured as follows. A sensitivity roll-off test is performed on a non-invasive blood glucose monitoring apparatus, and a signal intensity attenuation per unit depth is calculated through Gaussian fitting in the depth direction as the attenuation parameter σ.

In the above operation, the three-dimensional optical scattering coefficient of the target region is calculated from an OCT depth attenuation signal:

$$μ ( x , y , z ) = \frac{I ( x , y , z )}{2 Δ zn {\sum }_{z + 1}^{∞} I ( x , y , z )}$$

where, x denotes a fast scan direction in OCT three-dimensional scanning; y denotes a slow scan direction in OCT three-dimensional scanning; z denotes a depth direction in OCT three-dimensional scanning. The depth direction is perpendicular to a plane formed by the fast scan direction and the slow scan direction. μ(x, y, z) denotes an optical scattering coefficients at pixel position (x, y, z). l(x, y, z) denotes a compensated OCT signal intensity at the pixel position (x, y, z). n denotes a refractive index of the tested tissue region. Δz denotes an air-equivalent physical dimension per pixel in the depth direction.
S22, three-dimensional microvascular distribution of the target tissue region is generated from the OCT signal.
S22, the three-dimensional microvascular distribution of the target tissue region from the OCT signal is specifically generated as follows. OCT signal amplitude or phase, or both amplitude and phase are analyzed by an OCT blood flow signal extraction method to obtain the three-dimensional microvascular distribution of the target tissue region.

The OCT blood flow signal extraction method includes: difference calculation, speckle variance operation, decorrelation calculation, or eigenvalue decomposition calculation.
S23, the three-dimensional microvascular distribution is subjected to image binarization to obtain a vascular mask and a tissue mask, and the three-dimensional optical scattering coefficient is point-wise multiplied with the vascular mask and the tissue mask separately to obtain the BOC and the TOC.

Specifically, the S23 that the three-dimensional microvascular distribution is subjected to image binarization to obtain a vascular mask and a tissue mask, and the three-dimensional optical scattering coefficient is point-wise multiplied with the vascular mask and the tissue mask separately to obtain the BOC and the TOC is performed as follows.

A suitable threshold is selected to perform binarization on the three-dimensional microvascular distribution (i.e., a three-dimensional microvascular matrix). Specifically, pixel values of all blood flow regions in the three-dimensional microvascular distribution are set to 1, and pixel values of non-blood-flow regions are set to 0. The vascular mask is obtained, and the vascular mask is point-wise multiplied with the three-dimensional optical scattering coefficient to extract a blood flow optical scattering coefficient.

Pixel values of all blood flow regions in the three-dimensional microvascular distribution are set to 0, and pixel values of non-blood-flow regions are set to 1. The tissue mask is obtained, and the tissue mask is point-wise multiplied with the three-dimensional optical scattering coefficient to extract the TOC.

BGC calibration and calculation method 3 includes the following steps. A blood sample is collected, and reference BGCs at two time points t1 and t2 are rapidly measured through an electrochemical method. It is recommended that t1 and t2 represent pre-meal and post-meal times, respectively, and sampling frequency can be increased to improve calibration accuracy. The reference BGCs and BOC at the same time points are paired and linearly fitted. A first fitting equation satisfies: BGC=a*BOC+b. First and second fitting parameters a and b are obtained, thereby obtaining reference BGCs at all time points.

Specifically, at the pre-meal and post-meal time points t1 and t2, a commercial blood glucose meter or other means is used to measure BGC as reference BGCs. The OCTA-measured BOCs and reference BGCs at the same time points are paired and linearly fitted to obtain a linear relationship: BGC=a*BOC+b, thereby obtaining fitting parameters a and b. The actual BGC is mapped based on the established linear relationship based on the OCTA-measured BOC.

Interstitial fluid glucose concentration calibration and calculation method 4 includes the following steps. Peak time difference ΔT between a BOC and a TOC is determined. Reference BGCs at two time points t1 and t2 are paired and linearly fitted with TOCs delayed by ΔT. A second fitting equation satisfies: IGC=c*TOC+d. Third and fourth fitting parameters c and d are obtained, thereby obtaining IGCs at all time points.

Specifically, delay time ΔT of the IGC relative to the BGC is calculated based on peak times of post-meal curves of the BOC and TOC. The reference BGCs at the two time points t1 and t2 are respectively paired and linearly fitted with TOCs delayed by ΔT to establish a linear relationship: IGC=c*TOC+d, thereby obtaining fitting parameters c and d. The IGC is mapped through the established linear relationship based on the OCTA-measured TOC.

In the embodiment of the present disclosure, calibration for the BGC and IGC of an individual is implemented through an OGTT.

The OGTT is performed on the subject individual. Reference BGCs are rapidly measured using a chemical method at the baseline time (t1) and 20 min after ingestion of a standard glucose solution (t2). The reference BGC and BOC at the same time point are paired and linearly fitted to obtain fitting parameters a and b, thereby obtaining BGCs at all time points.

An OCTA apparatus is used to continuously monitor the BOC and TOC of the subject individual undergoing the OGTT, thereby obtaining the peak time difference ΔT. The reference BGCs at the baseline time (t1) and 20 min after ingestion of the standard glucose solution (t2) are respectively paired and linearly fitted with TOCs delayed by ΔT, and fitting parameters c and d are obtained, thereby obtaining IGCs at all the time points.

FIG. 2 is a schematic diagram of a continuous non-invasive OCTA-based blood glucose test apparatus of the present disclosure. The main structure of a low-coherence interferometry (LCI) part of the apparatus is an interferometer, including light source 11, sample arm objective lens 17, interference signal detection apparatus 19, and polarization controller 24. Light emitted by the light source 11 is split into two beams by beam splitter 12. One beam enters a reference arm of the interferometer and is irradiated onto planar high-reflectivity mirror 14 through reference arm collimator 13. The other beam enters a sample arm, is collimated 15, reflected by the optical path, and focused onto a test sample. The sample 18 is placed at a focal plane of the sample arm objective lens 17. Light reflected back from the reference arm and the sample arm interferes and is received by the interference signal detection apparatus 19. For a fiber-based optical path, the polarization controller 24 is used to adjust the polarization state of the light beam so as to maximize signal interference.

Depending on different methods of detecting low-coherence interference signals, the continuous non-invasive OCTA-based blood glucose test apparatus shown in FIG. 2 specifically includes: a time-domain measurement apparatus, a spectral-domain measurement apparatus, and a swept-source measurement apparatus.
1) For the time-domain measurement apparatus, the light source 11 uses broadband low-coherence light, the planar mirror 14 is movable along an optical axis direction, and the interference signal detection apparatus 19 is a point detector. The optical path length of the reference arm is changed by moving the planar mirror 14, and interference signals from both arms are detected by the point detector 19. Low-coherence interference test is performed on scattering signals in the depth direction z at a specific spatial depth to obtain a sampling volume in the spatial dimension of depth.
2) For the spectral-domain measurement apparatus, the light source 11 uses broadband low-coherence light, the planar mirror 14 remains fixed, and the interference signal detection apparatus 19 employs a spectrometer. When an interference signal is transmitted through a line-scan camera in the spectrometer, an interference spectrum is simultaneously recorded. Fourier analysis is applied to analyze the interference spectral signal and obtain scattering information in the depth direction z, thereby obtaining a sampling volume in the dimension space of depth.
3) For the swept-source measurement apparatus, the light source 11 uses a swept-source, the planar mirror 14 remains fixed, and the interference signal detection apparatus 19 employs a point detector. The point detector records a low-coherence interference spectrum of the swept-source in a time-division manner. Fourier analysis is applied to the interference spectral signal to obtain scattering information in the depth direction z, thereby obtaining a sampling volume in the dimension space of depth.

For the aforementioned different measurement apparatuses, the OCTA method involved in FIG. 1 can be combined to perform noise suppression and defect compensation, and to enhance real-time performance and spatial correspondence of the measurement.

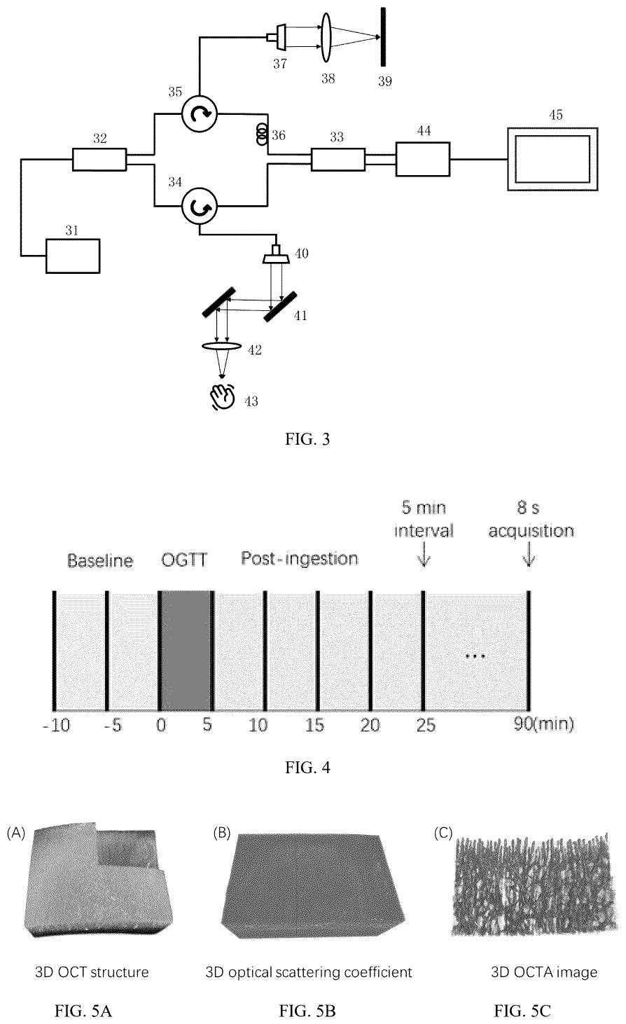

FIG. 3 is a schematic diagram of an exemplary embodiment of the present disclosure. The continuous non-invasive OCT blood glucose test apparatus includes broadband swept-source 31, 80:20 fiber coupler 32, 50:50 fiber coupler 33, first optical circulator 34, second optical circulator 35, polarization controller 36, first collimator 40, second collimator 37, focusing lens 38, scanning lens 42, planar high-reflectivity mirror 39, scanning galvanometer 41, sample 43, balanced detector 44, and processor 45. Light emitted by the broadband swept-source 31 used in the apparatus of the present disclosure enters the 80:20 fiber coupler 32 through an optical fiber. Light outgoing form the fiber coupler 32 is split into two beams. One beam enters the second collimator 37 through the second optical circulator 35. Collimated light exiting the second collimator 37 is focused by the focusing lens 38 and irradiated onto the planar high-reflectivity mirror 39. The other beam enters the first collimator 40 through the first optical circulator 34, passes through the scanning galvanometer 41 and the scanning lens 42, and is irradiated onto the sample 43. Light exiting the second optical circulator 35 passes through the polarization controller 36 and enters the 50:50 fiber coupler 33 together with light exiting the first optical circulator 34. Light exiting the fiber coupler 33 enters the balanced detector 44 for interference signal acquisition and test. Subsequently, the signal is sent to the processor 45, and combined with clock and trigger signals of the light source for further analysis and processing to obtain the interference spectral signal.

FIG. 4 shows a blood glucose test process in the embodiment of the present disclosure. The test lasts 90 min, with −10 to 0 min being a baseline period, 0 to 5 min being a glucose solution ingestion period, and 5 to 90 min being a blood glucose recovery period. During the test, OCTA signal acquisition is performed every 5 min using the test apparatus of the present disclosure to obtain the OCT signal of the target region for data processing. The BGC is measured every 10 min using an electrochemical method as a reference BGC.

FIG. 5A shows a three-dimensional OCT structural image of a human fingernail region in a human finger skin imaging experiment. FIG. 5B shows a three-dimensional optical scattering coefficient distribution map of the human fingernail region. FIG. 5C shows a three-dimensional OCTA image of the human fingernail region.

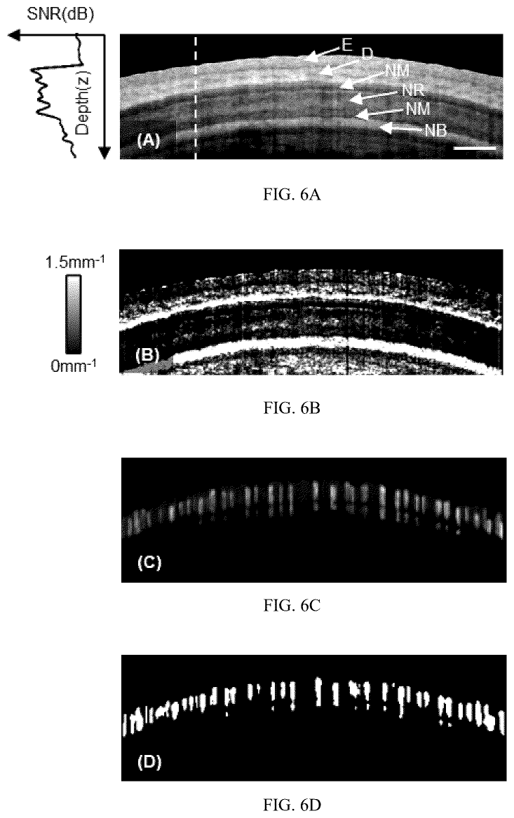

FIGS. 6A-6E show typical OCTA images in the embodiment of the present disclosure. FIG. 6A shows a cross-sectional image of the OCT structure in the target region, where the left curve shows an attenuation characteristic of the OCT signal intensity along the depth direction in the region indicated by white dashed lines in FIG. 6A. FIG. 6B shows a cross-sectional image of the optical scattering coefficient in the target region. FIG. 6C and FIG. 6D show cross-sectional images of OCTA and OCTA mask in the target region. FIG. 6E shows a maximum intensity projection of OCTA, where the white dashed line indicates the cross-sectional positions in FIG. 6A to FIG. 6D.

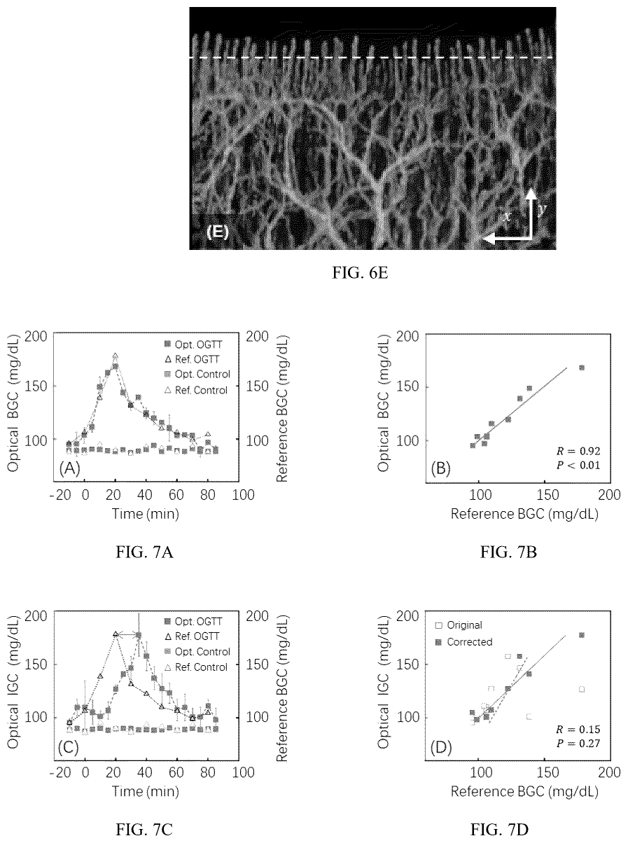

FIGS. 7A-7D show a relationship diagram of an optical BGC, an optical IGC, and a reference BGC versus time obtained by the method of the present disclosure during an OGTT in the embodiment of the present disclosure. FIG. 7A shows temporal variations of the optical BGC and the reference BGC during the OGTT, where the variations of the optical BGC and the reference BGC are completely synchronized without significant delay time. FIG. 7B shows correlation characterization between the optical BGC and the reference BGC, R=0.92. FIG. 7C shows temporal variations of the optical IGC and the reference BGC during the OGTT, where the optical IGC exhibits approximately a 15-min delay time relative to the reference BGC. FIG. 7D shows correlation characterization between the optical IGC and the reference BGC, where the correlation R value is 0.15 due to the delay time effect but increases to 0.89 after delay time correction.

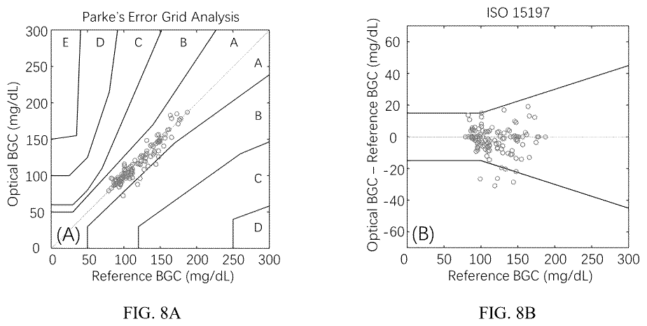

FIGS. 8A-8B show accuracy of non-invasive BGC measurement in the embodiment of the present disclosure. FIG. 8A shows Parke's error grid. Five risk zones are defined as: Zone A, clinically accurate; Zone B, benign; Zone C, overestimation; Zone D, undetectable; Zone E, erroneous. Both Zones A and B are clinically acceptable. All measured optical BGCs in the embodiment of the present disclosure fall within the clinically acceptable Zones A+B, with 96.69% of values in Zone A. This indicates that optical BGCs measured by the present disclosure are close to true BGC values in most cases. FIG. 8B shows a schematic diagram of an ISO 15197:2013 standard, where the upper and lower solid lines represent a ±15 mg/dL relative difference when the reference BGCs are less than 100 mg/dL, and represent a ±15% relative difference when the reference BGCs are not less than 100 mg/dL. The measured values within the two solid lines satisfy the ISO 15197:2013 standard. Among the measured optical BGCs in the embodiment of the present disclosure, 94.21% meet the ISO accuracy standard.

The above experimental results fully demonstrate that the continuous non-invasive optical BGC measurement can be achieved using the method and apparatus of the present disclosure. The test process is friendly and convenient, which provides enlightening significance for non-invasive blood glucose monitoring.

What is claimed is:

1. A non-invasive continuous blood glucose concentration (BGC) measurement method based on optical coherence tomography angiography (OCTA), comprising:
a continuous OCTA imaging method, comprising: performing, by an OCT apparatus, continuous OCT scanning and imaging on a target tissue region; and obtaining, by a processor, an OCT signal;
a calculation method for optical scattering coefficients of blood and a surrounding tissue, comprising: generating, by the processor, a three-dimensional optical scattering coefficient and a three-dimensional microvascular distribution of the target tissue region based on the OCT signal;
and separately calculating a blood optical scattering coefficient (BOC) and a tissue optical scattering coefficient (TOC) based on the three-dimensional optical scattering coefficient and the three-dimensional microvascular distribution;
a BGC calibration and calculation method, comprising: collecting a blood sample, and measuring reference BGCs at two time points of t1 and t2 through an electrochemical method; and
performing, by the processor, data pairing and linear fitting on the reference BGC and a BOC at a same time point, thereby obtaining BGCs at all the time points;
an interstitial fluid glucose concentration (IGC) calibration and calculation method, comprising: performing, by the processor, data pairing and linear fitting on the reference BGCs at the two time points of t1 and t2 and corresponding TOCs delayed by ΔT, thereby obtaining IGCs at all the time points; and
a non-invasive diabetes detection method, comprising: extracting, by the processor, features of an optical BGC curve and an optical IGC curve over time, respectively, based on the BGCs and the IGCs at all the time points, and detecting diabetes based on the features of the optical BGC curve and the optical IGC curve over time.

2. The non-invasive continuous BGC measurement method based on OCTA according to claim 1, wherein the continuous OCTA imaging method is selected from one of the following methods:
a time-domain OCT imaging method that alters an optical path length of a reference arm through scanning;
alternatively, a spectral-domain OCT imaging method that records a spectral interference signal through a spectrometer; and
alternatively, a swept-source OCT imaging method that records the spectral interference signal through a swept-source.

3. The non-invasive continuous BGC measurement method based on OCTA according to claim 1, wherein the calculation method for the optical scattering coefficients of the blood and the surrounding tissue comprises:
extracting, based on a depth attenuation characteristic of OCT, the three-dimensional optical scattering coefficient of the target tissue region from the OCT signal;
generating the three-dimensional microvascular distribution of the target tissue region from the OCT signal; and
subjecting the three-dimensional microvascular distribution to image binarization, and obtaining a vascular mask and a tissue mask; and subjecting the three-dimensional optical scattering coefficient to point-wise multiplication with the vascular mask and the tissue mask separately, and obtaining a BOC and a TOC.

4. The non-invasive continuous BGC measurement method based on OCTA according to claim 3, wherein the step of extracting, based on the depth attenuation characteristic of OCT, the three-dimensional optical scattering coefficient of the target tissue region from the OCT signal comprises:
first, removing system noise in a depth direction of the OCT signal, and obtaining a denoised OCT signal;
next, compensating a depth-direction attenuation in the denoised OCT signal through a Gaussian fitting model, and obtaining a compensated OCT signal; and
finally, calculating the three-dimensional optical scattering coefficient of the target tissue region according to a depth-direction variation feature of the compensated OCT signal.

5. The non-invasive continuous BGC measurement method based on OCTA according to claim 3, wherein the step of generating the three-dimensional microvascular distribution of the target tissue region from the OCT signal comprises: analyzing, by an OCT blood flow signal extraction method, OCT signal amplitude or phase, or both amplitude and phase, and obtaining the three-dimensional microvascular distribution of the target tissue region.

6. The non-invasive continuous BGC measurement method based on OCTA according to claim 5, wherein the OCT blood flow signal extraction method comprises: difference calculation, speckle variance operation, decorrelation calculation, or eigenvalue decomposition calculation.

7. The non-invasive continuous BGC measurement method based on OCTA according to claim 3, wherein the step of subjecting the three-dimensional microvascular distribution to image binarization, and obtaining the vascular mask and the tissue mask; and subjecting the three-dimensional optical scattering coefficient to point-wise multiplication with the vascular mask and the tissue mask separately, and obtaining the BOC and the TOC comprises:
setting pixel values of all blood flow regions in the three-dimensional microvascular distribution to 1 and pixel values of non-blood-flow regions to 0; obtaining the vascular mask; subjecting the vascular mask to point-wise multiplication with the three-dimensional optical scattering coefficient; and extracting the BOC; and
setting the pixel values of all blood flow regions in the three-dimensional microvascular distribution to 0 and the pixel values of the non-blood-flow regions to 1; obtaining the tissue mask; subjecting the tissue mask to point-wise multiplication with the three-dimensional optical scattering coefficient; and extracting the TOC.

8. A non-invasive continuous BGC measurement apparatus based on OCTA for implementing claim 1, comprising:
the OCT apparatus, configured to perform continuous OCT imaging on the target tissue region; and
one or more processors, configured to perform dynamic real-time data processing.

9. The non-invasive continuous BGC measurement apparatus based on OCTA according to claim 8, wherein the OCT apparatus is selected from one of:
an OCT apparatus comprising: a low-coherence broadband light source, an interferometer, and a point detector;
alternatively, an OCT apparatus comprising: the low-coherence broadband light source, the interferometer, and a spectrometer; and
alternatively, an OCT apparatus comprising: a swept broadband light source, the interferometer, and the point detector.

10. The non-invasive continuous BGC measurement apparatus based on OCTA according to claim 8, wherein in the non-invasive continuous BGC measurement method based on OCTA, the continuous OCTA imaging method is selected from one of the following methods:
a time-domain OCT imaging method that alters an optical path length of a reference arm through scanning;
alternatively, a spectral-domain OCT imaging method that records a spectral interference signal through a spectrometer; and
alternatively, a swept-source OCT imaging method that records the spectral interference signal through a swept-source.

11. The non-invasive continuous BGC measurement apparatus based on OCTA according to claim 8, wherein in the non-invasive continuous BGC measurement method based on OCTA, the calculation method for the optical scattering coefficients of the blood and the surrounding tissue comprises:
extracting, based on a depth attenuation characteristic of OCT, the three-dimensional optical scattering coefficient of the target tissue region from the OCT signal;
generating the three-dimensional microvascular distribution of the target tissue region from the OCT signal; and
subjecting the three-dimensional microvascular distribution to image binarization, and obtaining a vascular mask and a tissue mask; and subjecting the three-dimensional optical scattering coefficient to point-wise multiplication with the vascular mask and the tissue mask separately, and obtaining a BOC and a TOC.

12. The non-invasive continuous BGC measurement apparatus based on OCTA according to claim 11, wherein in the non-invasive continuous BGC measurement method based on OCTA, the step of extracting, based on the depth attenuation characteristic of OCT, the three-dimensional optical scattering coefficient of the target tissue region from the OCT signal comprises:
first, removing system noise in a depth direction of the OCT signal, and obtaining a denoised OCT signal;
next, compensating a depth-direction attenuation in the denoised OCT signal through a Gaussian fitting model, and obtaining a compensated OCT signal; and
finally, calculating the three-dimensional optical scattering coefficient of the target tissue region according to a depth-direction variation feature of the compensated OCT signal.

13. The non-invasive continuous BGC measurement apparatus based on OCTA according to claim 11, wherein in the non-invasive continuous BGC measurement method based on OCTA, the step of generating the three-dimensional microvascular distribution of the target tissue region from the OCT signal comprises: analyzing, by an OCT blood flow signal extraction method, OCT signal amplitude or phase, or both amplitude and phase, and obtaining the three-dimensional microvascular distribution of the target tissue region.

14. The non-invasive continuous BGC measurement apparatus based on OCTA according to claim 13, wherein in the non-invasive continuous BGC measurement method based on OCTA, the OCT blood flow signal extraction method comprises: difference calculation, speckle variance operation, decorrelation calculation, or eigenvalue decomposition calculation.

15. The non-invasive continuous BGC measurement apparatus based on OCTA according to claim 11, wherein in the non-invasive continuous BGC measurement method based on OCTA, the step of subjecting the three-dimensional microvascular distribution to image binarization, and obtaining the vascular mask and the tissue mask; and subjecting the three-dimensional optical scattering coefficient to point-wise multiplication with the vascular mask and the tissue mask separately, and obtaining the BOC and the TOC comprises:
setting pixel values of all blood flow regions in the three-dimensional microvascular distribution to 1 and pixel values of non-blood-flow regions to 0; obtaining the vascular mask; subjecting the vascular mask to point-wise multiplication with the three-dimensional optical scattering coefficient; and extracting the BOC; and
setting the pixel values of all blood flow regions in the three-dimensional microvascular distribution to 0 and the pixel values of the non-blood-flow regions to 1; obtaining the tissue mask; subjecting the tissue mask to point-wise multiplication with the three-dimensional optical scattering coefficient; and extracting the TOC.

16. The non-invasive continuous BGC measurement apparatus based on OCTA according to claim 10, wherein the OCT apparatus is selected from one of:
an OCT apparatus comprising: a low-coherence broadband light source, an interferometer, and a point detector;
alternatively, an OCT apparatus comprising: the low-coherence broadband light source, the interferometer, and the spectrometer; and
alternatively, an OCT apparatus comprising: a swept broadband light source, the interferometer, and the point detector.

17. The non-invasive continuous BGC measurement apparatus based on OCTA according to claim 11, wherein the OCT apparatus is selected from one of:
an OCT apparatus comprising: a low-coherence broadband light source, an interferometer, and a point detector;
alternatively, an OCT apparatus comprising: the low-coherence broadband light source, the interferometer, and a spectrometer; and
alternatively, an OCT apparatus comprising: a swept broadband light source, the interferometer, and the point detector.

18. The non-invasive continuous BGC measurement apparatus based on OCTA according to claim 12, wherein the OCT apparatus is selected from one of:
an OCT apparatus comprising: a low-coherence broadband light source, an interferometer, and a point detector;
alternatively, an OCT apparatus comprising: the low-coherence broadband light source, the interferometer, and a spectrometer; and
alternatively, an OCT apparatus comprising: a swept broadband light source, the interferometer, and the point detector.

19. The non-invasive continuous BGC measurement apparatus based on OCTA according to claim 13, wherein the OCT apparatus is selected from one of:
an OCT apparatus comprising: a low-coherence broadband light source, an interferometer, and a point detector;
alternatively, an OCT apparatus comprising: the low-coherence broadband light source, the interferometer, and a spectrometer; and
alternatively, an OCT apparatus comprising: a swept broadband light source, the interferometer, and the point detector.

20. The non-invasive continuous BGC measurement apparatus based on OCTA according to claim 14, wherein the OCT apparatus is selected from one of:
an OCT apparatus comprising: a low-coherence broadband light source, an interferometer, and a point detector;
alternatively, an OCT apparatus comprising: the low-coherence broadband light source, the interferometer, and a spectrometer; and
alternatively, an OCT apparatus comprising: a swept broadband light source, the interferometer, and the point detector.
